(19) United States
(12) Patent Application Publication
(10) Pub. No.: US 2025/0228581 A1
(43) Pub. Date: Jul. 17, 2025

(54) ENDOVASCULAR THROMBUS SCRAPER AND RELATED METHODS

(71) Applicant: BARD PERIPHERAL VASCULAR, INC., Franklin Lakes, NJ (US)

(72) Inventors: Casey Rockwood, Tulsa, OK (US); Melissa Boyle, Phoenix, AZ (US); Sam Shreve, Scottsdale, AZ (US); Edgar Sanchez Garcia, Tempe, AZ (US); David Tze, Phoenix, AZ (US)

(21) Appl. No.: 18/700,398

(22) PCT Filed: Oct. 11, 2021
(86) PCT No.: PCT/US2021/054394

§ 371 Date: Apr. 11, 2024

(51) Int. Cl. A61B 17/221; A61B 17/00; A61B 17/22

(52) U.S. Cl. CPC A61B 17/221; A61B 17/00234; A61B 2017/00238; A61B 2017/00305; A61B 2017/00477; A61B 2017/00557; A61B 2017/00862; A61B 2017/00867; A61B 2017/22061; A61B 2017/22065

(57) ABSTRACT

An apparatus for engaging a thrombus in a vessel includes a catheter having a first expandable element. A plurality of scrapers attached at a distal end portion to the catheter extend proximally at least partially alongside the first expandable element. In one embodiment, a proximal end portion of the plurality of scrapers is unattached to the catheter and adapted to be inserted between the thrombus and the vessel. Additional embodiments and related methods are also disclosed.

### BACKGROUND OF THE INVENTION

An endovascular thrombectomy involves the removal of a thrombus (blood clot) from a blood vessel, usually under image guidance. A current thrombectomy solution involves using a mesh stent and a “push and fluff” technique. This involves pushing the mesh of the stent radially through the thrombus, and then using a retriever to retrieve the stent and, hence, the thrombus.

While effective, the pressing of a mesh stent radially through the thrombus may cause it to break up into smaller pieces and travel unfettered in the bloodstream. This may increase the risk for embolism. Rotating the mesh stent in an effort to aid in thrombus dislodgment may actually make the situation worse in terms of generating small pieces, and may not serve to fully dislodge pieces of the thrombus from the vessel in situations where they are pressed against the inner wall of the vessel as a result of the mesh expansion.

Accordingly, a need is identified for a solution to the foregoing problem, and perhaps others that have yet to be discovered or understood. The solution would provide an effective device for thrombectomy that dislodges and captures the thrombus while seeking to reduce the creation of small pieces that may travel through the bloodstream, and thereby reduce the risk of embolism.

### SUMMARY OF THE INVENTION

An object of the disclosure is to provide an effective device for thrombectomy that dislodges and captures the thrombus while seeking to reduce the creation of small pieces that may travel through the bloodstream, and thereby reduce the risk of embolism.

According to a first aspect of the disclosure, an apparatus for engaging a thrombus in a vessel is provided. The apparatus comprises a catheter having a first expandable element. The catheter further includes a plurality of scrapers attached at a distal end portion to the catheter and extending proximally at least partially alongside the first expandable element. A proximal end portion of the plurality of scrapers is unattached to the catheter and adapted to be inserted between the thrombus and the vessel.

In one embodiment, the first expandable element comprises an inflatable balloon. In this or other embodiments, the plurality of scrapers each comprise a shape memory material. Each of the plurality of scrapers may be longer than the first expandable element. The catheter may include a second expandable element distal of the first expandable element. Each of the plurality of scrapers may have a width greater than a thickness. A web may be provided for interconnecting the plurality of scrapers. In any case, each of the plurality of scrapers is adapted to pass into a space between the thrombus and the vessel without buckling.

According to a second aspect of the disclosure, an apparatus for engaging a thrombus in a vessel is provided. The apparatus comprises a catheter including a distal end portion having a first expandable element, the catheter including a plurality of scrapers extending at least partially alongside the first expandable element, a proximal end portion of the plurality of scrapers extending at least partially alongside a second expandable element proximal of the distal end portion, the plurality of scrapers forming a cage lacking any expandable element therein and adapted to capture the thrombus.

In one embodiment, a first end of each of the plurality of scrapers is attached to the first expandable element and a second end of each of the plurality of scrapers is attached to the second expandable element. Each of the plurality of scrapers has a width greater than a thickness. The first and second expandable elements may each comprise an inflatable balloon, and the plurality of scrapers may comprise a shape memory material. Each of the plurality of scrapers may be longer than the first expandable element, and may be adapted for insertion between the thrombus and the vessel without buckling.

Still a further aspect of the disclosure relates to an apparatus for engaging a thrombus in a vessel. The apparatus comprises a catheter including a distal end portion having an expandable element. The catheter includes a plurality of scrapers attached at one end portion to the distal end portion of the catheter and extending at least partially alongside the expandable element, a second opposite end portion of the plurality of scrapers being attached to an elastomeric collar connected to the catheter proximal of the expandable element.

In one embodiment, each of the plurality of scrapers has a width greater than a thickness. The expandable element may comprise an inflatable balloon. The plurality of scrapers may comprise a shape memory material. Each of the plurality of scrapers may be longer than the expandable element, and may be adapted to pass into a space between the thrombus and the vessel without buckling.

Still a further aspect of the disclosure relates to a method of engaging a thrombus in a vessel. The method comprises inserting a first end portion of each of a plurality of scrapers attached at a second end portion to a catheter in a space between the thrombus and the vessel. The method may further comprise rotating the catheter. Prior to the inserting step, the catheter may be advanced distally of the thrombus and the expandable element expanded. The method may further include the step of expanding a balloon distally of the second end portion of the catheter prior to the inserting step.

### BRIEF DESCRIPTION OF THE DRAWINGS

The above and further advantages of the present disclosure may be better understood by referring to the following description in conjunction with the accompanying drawings in which:

FIG. 1 is a perspective view of a balloon catheter.

FIG. 1A is a cross-sectional view along line 1A-1A of FIG. 1.

FIG. 2 is a partially cutaway, perspective view of an endovascular thrombus scraper according to one aspect of the disclosure.

FIG. 2A is a cross-sectional view of a thrombus scraper.

FIG. 3 is a partially cutaway, perspective view of the scraper according to FIG. 2 in an expanded condition, with the scrapers in a deployed position.

FIGS. 4, 5, 6, and 7 are progressive schematic views illustrating the manner in which the scraper of FIGS. 1 and 2 may be used during a thrombectomy.

FIGS. 8 and 9 illustrate a further embodiment of an endovascular thrombus scraper.

FIGS. 10 and 11 illustrate yet a further embodiment of an endovascular thrombus scraper.

FIGS. 12, 13, and 14 illustrate still further embodiments of an endovascular thrombus scraper.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

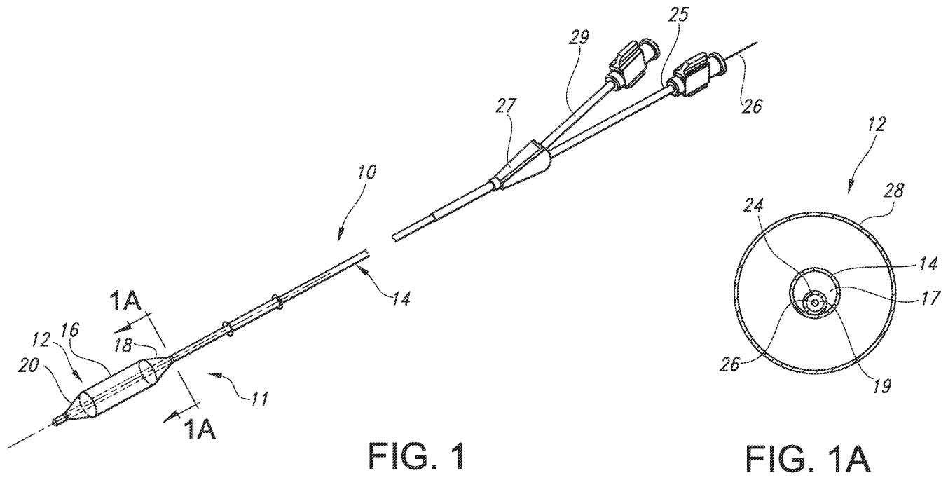

With reference to FIG. 1, provided is a catheter 10 having a distal end portion 11 including an expandable element in the form of a single balloon 12 mounted on a catheter shaft 14. The balloon 12 includes an intermediate portion 16, or “barrel,” and end portions 18, 20. In one embodiment, the end portions 18, 20 of the balloon 12 reduce in diameter or taper to join the intermediate portion 16 to the catheter shaft 14 (and thus tapered portions 18, 20 are generally termed cones or cone portions). The balloon 12 is sealed at balloon ends on the cone portions 18, 20 to allow the inflation of the balloon 12 via one or more inflation lumens 17 extending within catheter shaft 14 and communicating with the interior of the balloon 12.

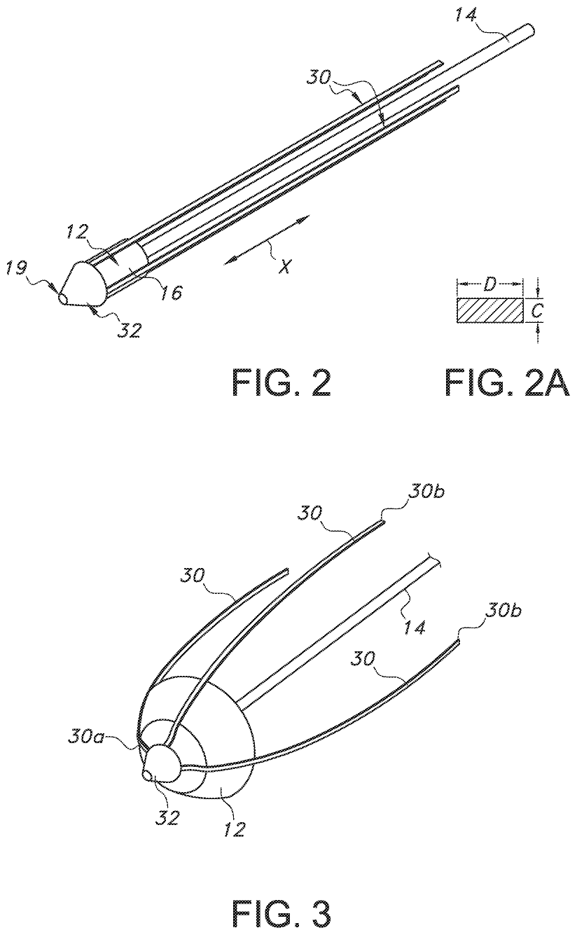

As indicated in FIG. 2, the catheter shaft 14 further includes an elongated, tubular shaft 24 at least partially nested therein. This internal shaft 24 includes a guidewire lumen 19 that directs a guidewire 26 through the catheter 10, including along the distal end portion 11 on which the single balloon 12 is located, and out through a tip of the catheter. As illustrated in FIG. 1, this guidewire 26 may extend through the proximal end portion of the catheter 10 and through a first port 25 of a connector or hub 27 into the guidewire lumen to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown). A second port 29 may also be associated with catheter 10, such as by way of connector or hub 27, for introducing a fluid (e.g., saline solution, a contrast agent, or both) into the interior compartment of the balloon 12 via the inflation lumen 17.

Inflatable balloon 12 may include a single or multi-layered balloon wall 28 forming the interior for receiving the inflation fluid. The balloon 12 may be a non-compliant balloon having a balloon wall 28 that maintains its size and shape in one or more directions when the balloon is inflated, and may comprise a plurality of inelastic fibers arranged in one or more layers. The balloon 12 in such case also has a pre-determined surface area that remains constant during and after inflation, also has a pre-determined length and pre-determined diameter that each, or together, remain constant during and after inflation. The balloon 12 (as well as any balloon described herein) can have any of a variety of diameters, ranging from 1-40 mm, such as 2.0-8.0 mm, and may have any of a variety of lengths ranging from 1-300 mm. As outlined further in the discussion that follows, more than one inflatable balloon may be associated with the catheter.

According to a first aspect of the disclosure, the catheter 10 may be adapted to dislodge, or scrape, a thrombus from within a vessel and capture the same for recovery. As illustrated in FIGS. 2 and 3, this may be achieved by providing a plurality of elongated scrapers for engaging the thrombus, which may take the form of fingers 30. The fingers 30 may be elongated in an axial direction X, and may be provided in a variety of widths, as outlined further in the following description. In the illustrated version, the fingers 30 have a thickness C less than its width D, as indicated in FIG. 2A.

Regardless of the number, shape, and size, the fingers 30 at a distal end 30a are attached to a tip 32 of the catheter 10 distal of the balloon 12 (which may be initially uninflated and folded), and through which tip the guidewire 26 may pass via lumen 19. The fingers 30 overlie and extend along the balloon 12, including the barrel portion 16 (which in FIG. 2 is shown as slightly inflated for illustration purposes). Thus, when the balloon 12 is fully expanded, as shown in FIG. 3, the fingers 30 are urged radially outwardly as a result (proximal portion of balloon removed for purposes of illustration). At the opposite, proximal portion or end 30b, the fingers 30 are free of attachment to the catheter 10, and extend proximally of the proximal end of the balloon 12 defined by the cone portion 18. Consequently, the fingers 30 are thus each longer than the balloon 12 by design.

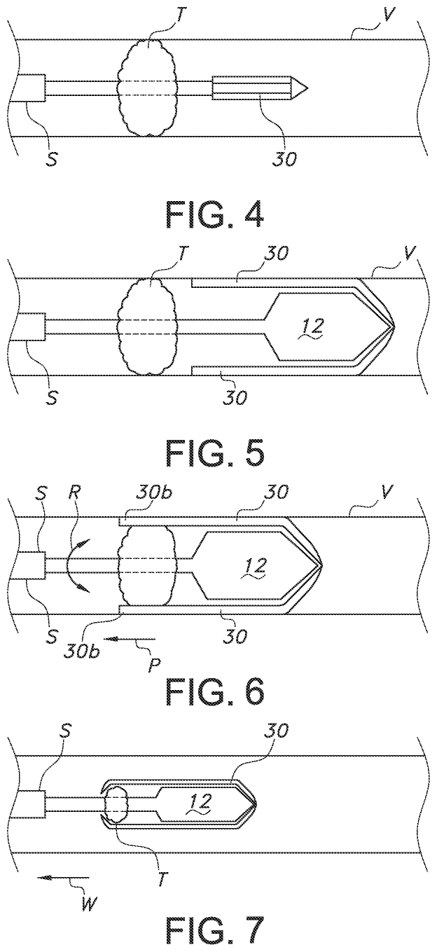

In use, and with reference to FIGS. 4, 5, 6, and 7, the catheter 10 may be advanced within a vessel V to the site of a thrombus T with the aid of a sheath S, which make take the form of an aspiration catheter. The catheter 10 is then advanced through the thrombus T, with the balloon 12 in an uninflated or low profile condition. Once the proximal ends of the scrapers or fingers 30 are fully past the thrombus T, the balloon 12 is inflated to assist in moving the scrapers or fingers 30 from a retracted position to an actuated or deployed position by pushing them radially outwardly, such as to touch the inner wall of the vessel V. The fingers 30 may also be formed of a shape memory material, such as Nitinol, and thus a temperature change may also be used (such as via fluid introduced to the interior of the vessel a certain temperature) to cause the scrapers to move outwardly toward and possibly into contact with the inner wall of the vessel V.

With the scrapers or fingers 30 deployed in this manner, the catheter 10 may then be drawn or pulled proximally, as indicated by arrow P. This movement causes the free ends 30b of the fingers 30 to enter into an interstitial space between the thrombus and the inner wall of the vessel V without buckling or folding, thus capturing the thrombus T between the fingers and also aiding in dislodging it from the vessel. Since the fingers 30 do not move radially within the thrombus T in this version, and only engage it peripherally, the thrombus is less likely to break into multiple small pieces.

As indicated by action arrow R, the catheter 10 may also be bodily rotated with the fingers 30 in the deployed position (such as to and fro to a limited degree, for example, but full rotation is of course possible). This rotation of the catheter 10 causes the deployed fingers 30 to move circumferentially along the inner wall of the vessel V and thereby potentially aid in dislodging and capturing the thrombus T. This movement may also be combined with the retraction or advance of the catheter 10 to aid in dislodgement and capture, as necessary for a particular situation.

As indicated in FIG. 7, the balloon 12 may be deflated as a further step in the thrombectomy. This deflation of the balloon 12 allows the fingers 30 to return to the non-deployed condition, possibly using the features of the shape memory material by causing a change in state. The deflated balloon 12 and non-deployed fingers 30 capturing the dislodged thrombus T may then be withdrawn via the aspiration catheter or sheath S (indicated by arrow W).

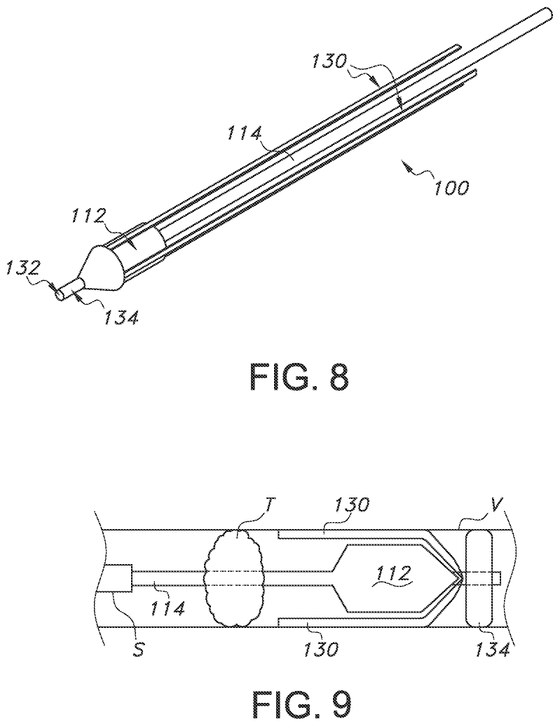

Turning to FIG. 8, a second embodiment of a catheter 100 is shown, which may include an initially uninflated balloon 134 adjacent the tip 132 at the distal end of the shaft 114. In use, this second balloon 134 may be inflated using a separate inflation lumen (not shown) during the above-described procedure to activate the fingers 130. This distal balloon 134 may thus stop any portion of the thrombus T from traveling past the inflated balloon, as shown in FIG. 9. By using a separate inflation lumen, the distal balloon 134 may remain inflated while balloon 112 is deflated and the fingers 130 close inwardly to capture the thrombus. Once captured, the distal balloon 134 may be independently deflated for withdrawal of the catheter 100.

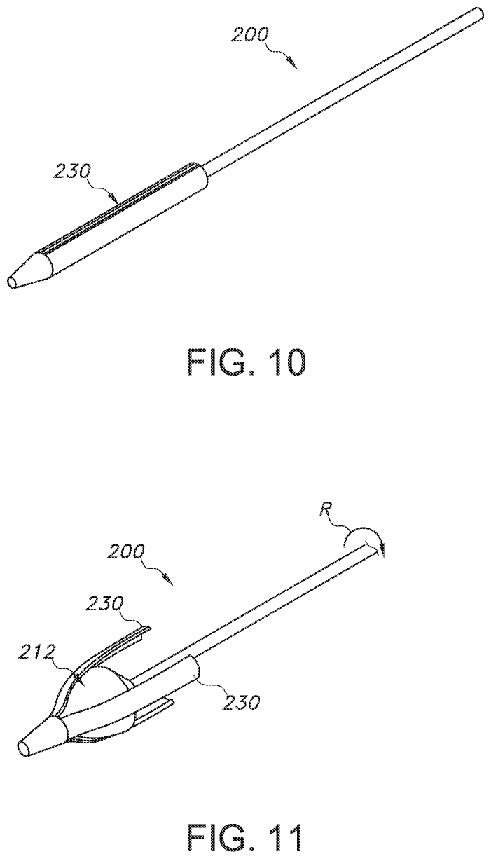

FIGS. 10 and 11 illustrate yet another embodiment of a catheter 200 adapted for performing a thrombectomy. As can be appreciated, FIG. 10 represents balloon in folded, uninflated condition, which balloon is only partially shown in FIG. 11. In this version, the scrapers take the form of fingers 230 wider in a radial direction and shorter in an axial direction that in the previously depicted versions (but still longer than the balloon 212). This tends to provide better columnar strength to the fingers 230 during rotation R, which may help to avoid any issues with folding or collapse during the thrombectomy.

In the above embodiments, the fingers 30, 130, 230 may be laser cut from a single tube. This provides each finger with a degree of curvature in a circumferential direction. Once formed, the free ends (e.g., proximal end 30b of finger 30) may be deburred or rounded to eliminate any sharp edges, thereby ensuring an atraumatic presentment along the inner wall of the vessel when the thrombus is engaged as previously described.

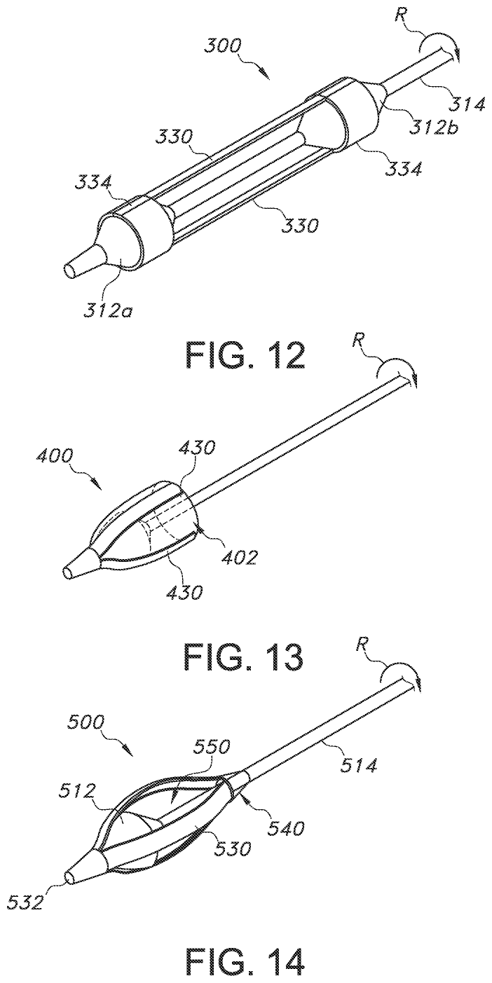

Still further embodiments of thrombectomy catheters are shown in FIGS. 12, 13, and 14. In FIG. 12, a catheter 300 includes a first, distal balloon 312a and a second, proximal balloon 312b spaced therefrom. A plurality of fingers 330 span between the balloons 312a, 312b, which may be inflated (via lumen) to move the fingers radially outwardly, as shown. The respective ends of the fingers 330 may be attached to or captured in place by expandable collars 334 over the barrel portion of each balloon 312a, 312b. In view of the full radial expansion caused by the balloons 312a, 312b, the fingers 330 in this version may be formed of a non-shape memory material, such as for example stainless steel, but could also be formed of a shape memory material.

In use, the catheter 300 may be advanced through a thrombus such that it lies between the uninflated balloons 312a, 312b. The balloons 312a, 312b may then be inflated to cause the fingers 330 to pass radially through the thrombus into an interstitial space with the vessel. This may serve to capture the thrombus between the fingers 330, but with minimal disruption (possibly only dividing the thrombus into two halves in the case of two fingers 330, as shown). The catheter 300 may also be rotated once the fingers 330 are expanded to assist in scraping and dislodgement of the thrombus, as indicated by arrow R.

In any case, individual pieces of a thrombus created as a result of using the catheter 300 for performing a thrombectomy are prevented from traveling upstream or downstream in the vessel by the inflated balloons 312a, 312b, and may potentially be aspirated through a port in the catheter shaft 314. The balloons 312a, 312b may be deflated to allow the fingers 330 to compress around and capture the thrombus (or portions thereof) for removal from the vessel, such as by an aspiration catheter (not shown).

FIG. 13 illustrates an embodiment of a thrombectomy catheter 400 in which a flexible material, such as a webbing 402 is provided to connect the fingers 430. This webbing 402 may also enter the space between the thrombus and vessel inner wall with fingers 430. Thus, the webbing 402, and may further aid in scraping and then capturing the thrombus when the fingers 430 retract as previously described (including via rotation R).

The webbing 402 may be expandable or elastic to allow the fingers 430 to expand outwardly as described above on inflation of the associated balloon. As examples, the webbing 402 may comprise a polymer sheet or film (such as, for example, PTFE, PET, Nylon, PEBAX or the like), but could also take the form of a polymer or metal mesh or other porous material. The webbing 402 may also extend to the free ends of the fingers 430, or may terminate proximally thereof.

In still a further embodiment of a catheter 500 adapted to perform a thrombectomy, which includes an expandable element, such as balloon 512, and fingers 530 connected at a distal end to a tip 532 of the catheter. The proximal ends of fingers 530 are connected to a connector, such as a collar 540 proximal of the balloon 512, which helps to prevent deflection during rotation of the catheter 500.

The collar 540 may comprise a flexible material, such as an elastomer. The collar 540 may be connected directly to the shaft 514, such as by welding or adhesive. The fingers 530 in this embodiment may be formed of a non-shape memory material, such as for example stainless steel, but could also be formed of a shape memory material.

In use, the catheter 500 once passed through a thrombus may be used to actuate or expand the balloon 512, thus pressing the fingers 530 radially outwardly to form a cage 550 for capturing the thrombus therein. Rotation of the catheter 500 (arrow R) may be done to scrape or dislodge the thrombus, as indicated by arrow R, which the collar 540 allowing for flexibility in the movement of fingers 530 to avoid causing damage to the vessel while allowing for movement into a space between the thrombus and the inner wall of the vessel to capture the former. The balloon 512 may then be deflated to capture the thrombus between the fingers 530, and the catheter 500 withdrawn to complete the thrombectomy.

Summarizing, this disclosure may be considered to relate to the following items:
1. An apparatus for engaging a thrombus in a vessel, comprising: a catheter having a first expandable element, the catheter further including a plurality of scrapers attached at a distal end portion to the catheter and extending proximally at least partially alongside the first expandable element, a proximal end portion of the plurality of scrapers being unattached to the catheter and adapted to be inserted between the thrombus and the vessel.
2. The apparatus of item 1, wherein the expandable element comprises an inflatable balloon.
3. The apparatus of item 1 or item 2, wherein the plurality of scrapers each comprise a shape memory material.
4. The apparatus of any of items 1-3, wherein each of the plurality of scrapers is longer than the first expandable element.
5. The apparatus of any of items 1-4, further including a second expandable element distal of the first expandable element.
6. The apparatus of any of items 1-5, wherein each of the plurality of scrapers has a width greater than a thickness.
7. The apparatus of any of items 1-6, further including a web interconnecting the plurality of scrapers.
8. The apparatus of any of items 1-7, wherein each of the plurality of scrapers is adapted to pass into a space between the thrombus and the vessel without buckling.
9. An apparatus for engaging a thrombus in a vessel, comprising:
   a catheter including a distal end portion having a first expandable element, the catheter including a plurality of scrapers extending at least partially alongside the first expandable element, a proximal end portion of the plurality of scrapers extending at least partially alongside a second expandable element proximal of the distal end portion, the plurality of scrapers forming a cage lacking any expandable element therein and adapted to capture the thrombus.
10. The apparatus of item 9, wherein a first end of each of the plurality of scrapers is attached to the first expandable element and a second end of each of the plurality of scrapers is attached to the second expandable element.
11. The apparatus of item 9 or item 10, wherein each of the plurality of scrapers has a width greater than a thickness.
12. The apparatus of any of items 9-11, wherein the first and second expandable elements each comprise an inflatable balloon.
13. The apparatus of any of items 9-12, wherein the plurality of scrapers comprise a shape memory material.
14. The apparatus of any of items 9-13, wherein each of the plurality of scrapers is longer than the first expandable element.
15. The apparatus of any of items 9-14, wherein each of the plurality of scrapers is adapted for insertion between the thrombus and the vessel without buckling.
16. An apparatus for engaging a thrombus in a vessel, comprising:
   a catheter including a distal end portion having an expandable element, the catheter including a plurality of scrapers attached at one end portion to the distal end portion of the catheter and extending at least partially alongside the expandable element, a second opposite end portion of the plurality of scrapers being attached to an elastomeric collar connected to the catheter proximal of the expandable element.
17. The apparatus of item 16, wherein each of the plurality of scrapers has a width greater than a thickness.
18. The apparatus of item 16 or item 17, wherein the expandable element comprises an inflatable balloon.
19. The apparatus of any of items 16-18, wherein the plurality of scrapers comprise a shape memory material.
20. The apparatus of any of items 16-19, wherein each of the plurality of scrapers is longer than the first expandable element.
21. The apparatus of any of items 16-20, wherein each of the plurality of scrapers is adapted to pass into a space between the thrombus and the vessel without buckling.
22. A method of engaging a thrombus in a vessel, comprising:
   inserting a first end portion of each of a plurality of scrapers attached at a second end portion to a catheter in a space between the thrombus and the vessel.
23. The method item 22, further including the step of rotating the catheter.
24. The method of item 22 or claim 23, wherein, prior to the inserting step, the catheter is advanced distally of the thrombus and the expandable element is expanded.
25. The method of any of items 22-24, further including the step of expanding a balloon distally of the second end portion of the catheter prior to the inserting step.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. An apparatus for engaging a thrombus in a vessel, comprising:
a catheter having a first expandable element, the catheter further including a plurality of scrapers attached at a distal end portion to the catheter and extending proximally at least partially alongside the first expandable element, a proximal end portion of the plurality of scrapers being unattached to the catheter and adapted to be inserted between the thrombus and the vessel.

2. The apparatus of claim 1, wherein the first expandable element comprises an inflatable balloon.

3. The apparatus of claim 1, wherein the plurality of scrapers each comprise a shape memory material.

4. The apparatus of claim 1, wherein each of the plurality of scrapers is longer than the first expandable element.

5. The apparatus of claim 1, further including a second expandable element distal of the first expandable element.

6. The apparatus of claim 1, wherein each of the plurality of scrapers has a width greater than a thickness.

7. The apparatus of claim 1, further including a web interconnecting the plurality of scrapers.

8. The apparatus of claim 1, wherein each of the plurality of scrapers is adapted to pass into a space between the thrombus and the vessel without buckling.

9. An apparatus for engaging a thrombus in a vessel, comprising:
a catheter including a distal end portion having a first expandable element, the catheter including a plurality of scrapers extending at least partially alongside the first expandable element, a proximal end portion of the plurality of scrapers extending at least partially alongside a second expandable element proximal of the distal end portion, the plurality of scrapers forming a cage lacking any expandable element therein and adapted to capture the thrombus.

10. The apparatus of claim 9, wherein a first end of each of the plurality of scrapers is attached to the first expandable element and a second end of each of the plurality of scrapers is attached to the second expandable element.

11. The apparatus of claim 9, wherein each of the plurality of scrapers has a width greater than a thickness.

12. The apparatus of claim 9, wherein the first and second expandable elements each comprise an inflatable balloon.

13. The apparatus of claim 9, wherein the plurality of scrapers comprise a shape memory material.

14. The apparatus of claim 9, wherein each of the plurality of scrapers is longer than the first expandable element or the second expandable element.

15. The apparatus of claim 9, wherein each of the plurality of scrapers is adapted for insertion between the thrombus and the vessel without buckling.

16. An apparatus for engaging a thrombus in a vessel, comprising:
a catheter including a distal end portion having an expandable element, the catheter including a plurality of scrapers attached at one end portion to the distal end portion of the catheter and extending at least partially alongside the expandable element, a second opposite end portion of the plurality of scrapers being attached to an elastomeric collar connected to the catheter proximal of the expandable element.

17. The apparatus of claim 16, wherein each of the plurality of scrapers has a width greater than a thickness.

18. The apparatus of claim 16, wherein the expandable element comprises an inflatable balloon.

19. The apparatus of claim 16, wherein the plurality of scrapers comprise a shape memory material.

20. The apparatus of claim 16, wherein each of the plurality of scrapers is longer than the expandable element.

21. The apparatus of claim 16, wherein each of the plurality of scrapers is adapted to pass into a space between the thrombus and the vessel without buckling.

22.-25. (canceled)
